(19) United States
(12) Patent Application Publication
(10) Pub. No.: US 2020/0183243 A1
(43) Pub. Date: Jun. 11, 2020

(54) ACTIVE ELECTROCHROMIC FILMS

(71) Applicant: University of Utah Research Foundation, Salt Lake City, UT (US)

(72) Inventors: Logan Kiefer, Salt Lake City, UT (US); Max Gallant, Salt Lake City, UT (US); Nicholas Flinner, Salt Lake City, UT (US); Christian Robert, Salt Lake City, UT (US); Thomas Folke, Salt Lake City, UT (US); Taylor D. Sparks, Salt Lake City, UT (US)

(21) Appl. No.: 16/319,432

(22) PCT Filed: Jul. 20, 2017
(86) PCT No.: PCT/US17/43147

§ 371 Date: Jan. 21, 2019

Related U.S. Application Data
(60) Provisional application No. 62/364,836, filed on Jul. 20, 2016.

(51) Int. Cl. G02F 1/1503; G02F 1/155; C09K 9/02; C09B 57/00; C08F 20/14; C08K 5/13; C08K 5/3432; A61F 9/02; A42B 3/22; E06B 9/24

(52) U.S. Cl. CPC G02F 1/1503; G02F 1/155; C09K 9/02; C09B 57/00; C08F 20/14; E06B 2009/2464; C08K 5/3432; A61F 9/023; A42B 3/226; E06B 9/24; C09K 2211/1018; C08K 5/13

(57) ABSTRACT

An active electrochromic film (500) can include a transparent flexible substrate (510), a first transparent electrically conductive layer (520) in contact with the transparent flexible substrate (510), an active electrochromic gel layer (530) in contact with the transparent electrically conductive layer (520), and a second transparent electrically conductive layer (515) in contact with the active electrochromic gel layer (530) opposite from the first transparent electrically conductive layer (520). The active electrochromic gel layer (530) can include a viologen-based compound. The active electrochromic gel layer (530) can exhibit a high visible optical transparency in the absence of a voltage applied across the first and second transparent electrically conductive layers (520,515) and a low visible optical transparency under an applied voltage. In one example, at least one of the first and second transparent electrically conductive layers (520,515) can include a masked portion (580) such that the active electrochromic gel layer (530) is insulated from the applied voltage in an area adjacent to the masked portion (580).

### RELATED APPLICATION

This application claims priority to U.S. Provisional Patent Application No. 62/364,836 filed Jul. 20, 2016 which is incorporated herein by reference.

### BACKGROUND

In many modern residential and commercial buildings, poor energy efficiency of windows contributes significantly to high heating and cooling loads. Windows can be inherently less effective at containing heat because their primary purpose is to allow visible light in and out of the building. Typically, a window's efficiency is quantified by the Solar Heat Gain Coefficient (SHGC) which represents the fraction of solar energy allowed inside.

Dynamic tinting has been investigated as a means to reduce the amount of visible light transmitted by windows. Solar energy is made up of approximately 40% visible light. Therefore, a temporary reduction in the visual light transmission of a window pane by 80% can reduce the SHGC by 0.32. Electrochromic windows in particular have been investigated to reduce visible light transmission of windows. A study conducted by the National Renewable Energy Laboratory (NREL) suggests that the use of electrochromic windows in the place of all static tint windows in residential buildings could result in 13.5% electricity savings. However, electrochromic devices have often been complicated and expensive. Accordingly, research continues in the area of electrochromic technologies.

### SUMMARY

In one example of the present technology, an active electrochromic film can include a transparent flexible substrate, a first transparent electrically conductive layer in contact with the transparent flexible substrate, an active electrochromic gel layer in contact with the transparent electrically conductive layer, and a second transparent electrically conductive layer in contact with the active electrochromic gel layer opposite from the first transparent electrically conductive layer. The active electrochromic gel layer can include a viologen-based compound and exhibit a high visible optical transparency in the absence of a voltage applied across the first and second transparent electrically conductive layers and a low visible optical transparency under the applied voltage. Additionally, at least one of the first and second transparent electrically conductive layers can have a masked portion such that the active electrochromic gel layer is insulated from the applied voltage in an area adjacent to the masked portion.

In another example, an active electrochromic film can include a transparent flexible substrate, a first transparent electrically conductive layer in contact with the transparent flexible substrate, an active electrochromic gel layer in contact with the transparent electrically conductive layer, and a second electrically conductive layer in contact with the active electrochromic gel layer opposite from the first transparent electrically conductive layer. The active electrochromic gel layer can be a homogeneous gel including a solvent, a gel-forming polymer, and a viologen-based compound.

In a further example, a method of making an active electrochromic film can include pressing an active electrochromic gel composition between a first transparent electrically conductive layer and a second transparent electrically conductive layer. The first and second transparent electrically conductive layers can be in the form of roll-fed flexible materials. The active electrochromic gel composition can include a viologen-based compound.

Additional features and advantages of these principles will be apparent from the following detailed description, which illustrates, by way of example, features of the invention.

### BRIEF DESCRIPTION OF THE DRAWINGS

FIG. 1 is a side cross-sectional view of an active electrochromic film in accordance with an example of the present technology.

FIG. 2 is a side cross-sectional view of an active electrochromic film in accordance with an example of the present technology.

FIG. 3 is a side cross-sectional view of an active electrochromic film in accordance with an example of the present technology.

FIG. 4 is a side cross-sectional view of an active electrochromic film in accordance with an example of the present technology.

FIG. 5A is a side cross-sectional view of an active electrochromic film in accordance with an example of the present technology.

FIG. 5B is a top cross-sectional view of the active electrochromic film of FIG. 5A.

FIG. 6A is a side cross-sectional view of an active electrochromic film in accordance with an example of the present technology.

FIG. 6B is a top cross-sectional view of the active electrochromic film of FIG. 6A.

FIG. 7A is a side cross-sectional view of an active electrochromic film in accordance with an example of the present technology.

FIG. 7B is a top cross-sectional view of the active electrochromic film of FIG. 7A.

FIG. 8A is a perspective view of a motorcycle helmet visor having an applied active electrochromic film in accordance with an example of the present technology, in which the active electrochromic film is in a clear state.

FIG. 8B is a perspective view of the motorcycle helmet visor of FIG. 8A in which the active electrochromic film is in a dark state.

FIG. 9 is a schematic of a system for making an active electrochromic film in accordance with an example of the present technology.

FIG. 10 is a graph of % transmittance vs. light wavelength for films made using polyvinyl formal (PVF) and poly(methyl methacrylate) (PMMA).

FIG. 11 is a graph of % transmittance vs. time for the films made using PVF and PMMA.

It should be noted that the figures are merely exemplary of several embodiments and no limitations on the scope of the present invention are intended thereby. Further, the figures are generally not drawn to scale, but are drafted for purposes of convenience and clarity in illustrating various aspects of the invention.

### DETAILED DESCRIPTION

Reference will now be made to exemplary embodiments and specific language will be used herein to describe the same. It will nevertheless be understood that no limitation of the scope of the invention is thereby intended. Alterations and further modifications of the inventive features described herein, and additional applications of the principles of the invention as described herein, which would occur to one skilled in the relevant art and having possession of this disclosure, are to be considered within the scope of the invention. Further, before particular embodiments are disclosed and described, it is to be understood that this invention is not limited to the particular process and materials disclosed herein as such may vary to some degree. It is also to be understood that the terminology used herein is used for the purpose of describing particular embodiments only and is not intended to be limiting, as the scope of the present invention will be defined only by the appended claims and equivalents thereof.

Definitions

In describing and claiming the present invention, the following terminology will be used.

The singular forms “a,” “an,” and “the” include plural references unless the context clearly dictates otherwise. Thus, for example, reference to “a layer” includes reference to one or more of such structures, “a polymer” includes reference to one or more of such materials, and “applying” refers to one or more of such steps.

As used herein, “electrochromic” refers to a property allowing certain materials to change color when an electric charge is applied. An “active electrochromic” material can change color when a constant power supply is connected to the material and then revert back to the original color when the power supply is disconnected. A “passive electrochromic” material can change color and maintain that color even if the power supply is disconnected. Thus, a passive electrochromic material can require power only to switch between color states, and not to maintain either color state.

As used herein, “transparency” refers to the percentage of incident light that is transmitted through a material as opposed to being absorbed or reflected by the material. Thus, a material having a transparency of 70% allows 70% of incident light to pass through. The term “visible optical transparency” refers to the percentage of visible wavelengths of light that are transmitted through a material. As used herein, “transparent” refers to materials that transmit a majority of visible light, such as at least 70% of incident visible light. As used herein, “opacity” is the opposite of transparency, or the percentage of light that is not transmitted by a material. A material that is “more opaque” is understood to have a lower transparency, whereas a material that is “less opaque” has a higher transparency.

As used herein, “masking” and “masked” refers to the placement of a layer of material over a portion of surface area of a substrate to selectively separate that portion of the substrate from another layer that is deposited over the masking layer. For example, a masking material can be placed over a portion of a transparent electrode to mask the portion of the electrode before an electrochromic gel is deposited over the entire surface, electrode, and the masking material. The masking material can remain in place to electrically insulate the electrochromic gel from the electrode in that specific masked area. In another example, masking material can be placed on a polymer substrate before depositing a transparent conductive material to form an electrode. After forming the electrode, the masking material can be removed with the overlying conductive material, leaving behind an area without any conductive material. This type of process can be used, for example, to create multiple separate electrodes from a single layer of deposited transparent conductive material. Thus, masking processes can involve removing masking material and overlying material, or leaving the masking material in place (for example, to electrically insulate two layers one from another).

As used herein, “gel” refers to a jelly-like material that includes a polymer network with a dispersed liquid phase therein.

As used herein, “homogeneous” is used to refer to gels that do not include particles of other materials that are not a part of the gel. For example, a homogeneous gel may not include air bubbles, nanoparticles, or larger particles of solid materials such as carbon nanoparticles, graphite, metals particles, and so on. However, a homogeneous gel can include the solid gel-forming polymer that makes up the polymer network of the gel, one or more liquids making up the dispersed liquid phase of the gel, and soluble materials dissolved in the one or more liquids.

As used herein, “substantial” when used in reference to a quantity or amount of a material, or a specific characteristic thereof, refers to an amount that is sufficient to provide an effect that the material or characteristic was intended to provide. The exact degree of deviation allowable may in some cases depend on the specific context. Similarly, “substantially free of” or the like refers to the lack of an identified element or agent in a composition. Particularly, elements that are identified as being “substantially free of” are either completely absent from the composition, or are included only in amounts which are small enough so as to have no measurable effect on the composition.

As used herein, “about” refers to a degree of deviation based on experimental error typical for the particular property identified. The latitude provided the term “about” will depend on the specific context and particular property and can be readily discerned by those skilled in the art. The term “about” is not intended to either expand or limit the degree of equivalents which may otherwise be afforded a particular value. Further, unless otherwise stated, the term “about” shall expressly include “exactly,” consistent with the discussion below regarding ranges and numerical data. Unless otherwise enunciated, the term “about” generally connotes flexibility of less than 5%, and most often less than 1%, and in some cases less than 0.01%.

Concentrations, dimensions, amounts, and other numerical data may be presented herein in a range format. It is to be understood that such range format is used merely for convenience and brevity and should be interpreted flexibly to include not only the numerical values explicitly recited as the limits of the range, but also to include all the individual numerical values or sub-ranges encompassed within that range as if each numerical value and sub-range is explicitly recited. For example, a range of about 1 to about 200 should be interpreted to include not only the explicitly recited limits of 1 and 200, but also to include individual sizes such as 2, 3, 4, and sub-ranges such as 10 to 50, 20 to 100, etc.

As used herein, a plurality of items, structural elements, compositional elements, and/or materials may be presented in a common list for convenience. However, these lists should be construed as though each member of the list is individually identified as a separate and unique member. Thus, no individual member of such list should be construed as a de facto equivalent of any other member of the same list solely based on their presentation in a common group without indications to the contrary.

Active Electrochromic Films

The present technology provides electrochromic films that can be switched between various opacities using a small applied voltage. In some examples, the electrochromic films can be flexible and laminable, making the films appropriate for a wide variety of applications. For example, the electrochromic films can be laminated onto existing windows of homes or commercial buildings. Many existing electrochromic window technologies incorporate rigid glass substrates. Such electrochromic windows may be appropriate for replacement of existing windows, but do not allow for retrofitting existing windows. The present technology can reduce the cost of incorporating electrochromic functions into existing windows by allowing the existing windows to be retrofitted with a less expensive flexible film. Windows retrofitted with these films can dynamically block out a portion of sunlight transmitted by the window, which can lead to significant reductions in energy consumption to cool homes and commercial buildings.

In other examples, the flexible electrochromic films provided herein can be applied to a variety of transparent surfaces such as car windshields, sports goggles, motorcycle visors, and so on. Flexible films can conform to a variety of curved surfaces. In some cases, the electrochromic films can dynamically block a portion of sunlight to reduce glare in situations such as when a driver drives a car toward the setting sun.

One of the persistent problems preventing the widespread use of electrochromic window technology is the typically high price point. Many existing electrochromic windows require expensive manufacturing steps. For example, some electrochromic window technologies use a thin film of metal oxide as the electrochromic material. The metal oxide film can be formed by high vacuum sputtering, which is expensive and difficult to scale. In contrast, the present films use an active electrochromic gel layer that can be made by simply mixing the appropriate ingredients. The gel can then be pressed between transparent electrodes to form an electrochromic film, e.g. via hot pressing. The simple and inexpensive manufacturing steps used the make the present films can provide a cheap alternative to existing electrochromic window technologies.

Additionally, the laminable electrochromic films provided herein do not preclude the use of other coatings on windows. In existing electrochromic windows, which can be installed as window replacements rather than window augmentations, other coatings, such as low-e coatings or IR reflective coatings are present only if the manufacturer has chosen to include them. The laminable electrochromic films provided herein can be applied to any type of window. Therefore, the films can be added to windows with any combination of pre-existing energy efficiency features or coatings.

The active electrochromic films described herein can include a viologen-based compound as the electrochromic material. Viologen refers to bipyridinium derivatives of 4,4′-bipyridyl having the following general formula, in which R₁ and R₂ can be alkyl, cycloalkyl, aryl:

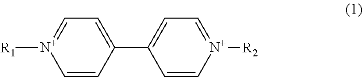

Viologen-based compounds can be reduced to a radical mono cation typically having a dark blue color, although other colors can be produced depending on the specific viologen. In the electrochromic films described herein, gels incorporating viologen-based compounds can be switched from a nearly transparent state to a dark blue state by applying a small voltage to the gels. Non-limiting examples of viologen-based compounds include methyl viologen, ethyl viologen, benzyl viologen, and others. As viologen-based compounds are cationic, they can be often be included with anions such as chloride, bromide, iodide, perchlorate, and others. In further examples, extended viologens can be used, such as compounds containing multiple viologen repeat units and compounds in which the pyridine rings of the viologen are separated by additional π-conjugated groups.

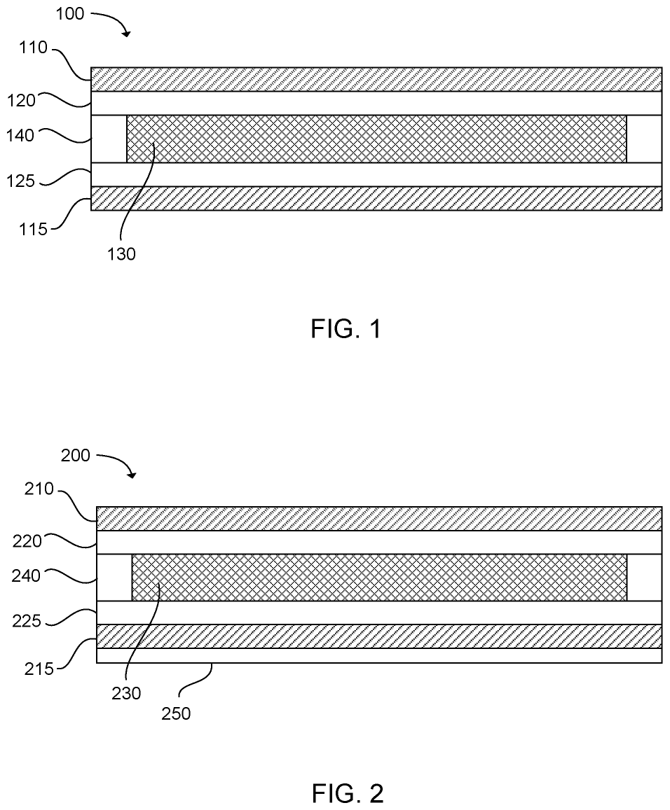

FIG. 1 shows a cross-sectional view of an example active electrochromic film 100 according to the present technology. The film can include: a transparent flexible substrate 110; a first transparent electrically conductive layer 120 in contact with the transparent flexible substrate; an active electrochromic gel layer 130 in contact with the transparent electrically conductive layer; a second transparent electrically conductive layer 125 in contact with the active electrochromic gel layer opposite from the first transparent electrically conductive layer; and a second transparent flexible substrate 115 in contact with the second transparent electrically conductive layer. A sealant 140 can be located between the first and second transparent electrically conductive layers and around a perimeter of the active electrochromic gel layer.

The active electrochromic gel layer can include a viologen-based compound as described above. Applying a voltage across the first and second transparent electrically conductive layers can reduce the viologen-based compound and turn the viologen to a darker color, such as a dark blue color. The darker color can cause the active electrochromic gel layer to have a lower visible optical transparency. Thus, voltage can be applied to lower the transparency of the active electrochromic gel layer and the voltage can be removed to increase the transparency of the layer.

In some examples, the active electrochromic gel layer can consist of a homogeneous gel. The gel can include a solvent, a gel-forming polymer, and the viologen-based compound. The homogeneous gel can be free of other materials that are not a part of the gel or dissolved in the solvent. Specifically, the homogeneous gel can be free of solid particles of other materials such as carbon nanoparticles, graphite, metal particles, and so on. Additionally, the active electrochromic gel layer can contact both the first and second transparent electrically conductive layers. Thus, in some examples the active electrochromic gel layer can be the only material between the transparent electrically conductive layers. In further examples, the active electrochromic gel layer can be the only material between the transparent electrically conductive layers with the exception of masking material and sealant.

In further examples, the solvent in the active electrochromic gel layer can be an organic solvent. In more particular examples, the solvent can be a polar organic solvent. Non-limiting examples of suitable solvents can include propylene carbonate, ethylene carbonate, diethyl carbonate, 2-pyrrolidone, N-methyl-2-pyrrolidone, dimethylformamide, dimethyl sulfoxide, sulfolane, or combinations thereof.

The amount of solvent in the active electrochromic gel layer can be varied to adjust the viscosity of the gel. In various examples, the solvent can be present in an amount of 92% to 72% with respect to the total weight of the active electrochromic gel layer. In further examples, the solvent can be present in an amount of 80% to 84% with respect to the total weight of the active electrochromic gel layer. The viscosity of the electrochromic gel layer can be 10,000 cp to about 100,000 cp.

The active electrochromic gel can be formed by dispersing the solvent with a gel-forming polymer. In certain examples, the gel-forming polymer can include poly(methyl methacrylate), poly(vinyl formal), or a combination thereof. Other non-limiting examples of gel-forming polymers can include poly(ethylene oxide), poly(acrylonitrile), poly(vinylidene fluoride), or combinations thereof. In various examples, the gel-forming polymer can be present in an amount of 5% to 30% with respect to the total weight of the active electrochromic gel layer. In further examples, the gel-forming polymer can be present in an amount of 14% to 24% (or from 10% to 20%) with respect to the total weight of the active electrochromic gel layer.

In some examples, the active electrochromic gel layer can include additional ingredients dissolved in the solvent. For example, anodic compounds such as, but not limited to hydroquinone, and the like can be used. Such anodic compounds form charge transfer complexes with the viologen aiding in electron transfer and increasing electrochromic transition rates. In a particular example, the active electrochromic gel layer can include hydroquinone. In certain embodiments, the active electrochromic gel layer can include hydroquinone in an amount of 0.01% to 0.81% with respect to the total weight of the active electrochromic gel layer. In further embodiments, the active electrochromic gel layer can include hydroquinone in an amount of 0.2% to 0.6% (and in one example about 0.33%) with respect to the total weight of the active electrochromic gel layer.

In further examples, the active electrochromic gel layer can be transparent when no electric current is applied to the layer and then become less transparent or opaque when an electric current is applied. For example, the active electrochromic gel layer can exhibit a high visible optical transparency of at least 70% in the absence of an applied voltage and a low visible optical transparency of less than 50% under the applied voltage. In other examples, the high visible optical transparency can be at least 80% and the low visible optical transparency can be less than 20%. Furthermore, in some examples the applied voltage can be varied to adjust the transparency of the layer. For example, a high voltage can be applied to produce a low transparency in the layer, while an intermediate voltage can be applied to produce an intermediate transparency in the layer. Thus, the transparency level of the layer can be dynamically tuned by varying the applied voltage. The voltages used can generally be modest, such as from 0V to 6V. In further examples, the applied voltage can be varied from 0V to 3V. The current used to maintain the active electrochromic gel layer in the low transparency state can also be modest. In some examples, the current required for a given area of the layer can be 1 A/m² to 50 A/m².

The active electrochromic films described herein can generally be made to have any desired dimensions. Films can be made with large surface areas to cover large windows, or with smaller surface areas to cover smaller articles such as motorcycle helmet visors or goggles. For large applications such as windows, films can have a length and width ranging from 10 cm to 10 m or more, for example. In smaller applications, films can have lengths and widths ranging from 1 cm to 10 cm, for example. The overall thickness of the films can be 0.1 mm to 2 mm and in some cases 0.2 mm to 1 mm.

In some examples, the active electrochromic gel layer in particular can have a thickness of 0.05 mm to 0.7 mm. As a general guideline, thinner layers tend to transition faster, while thicker layers tend to provide darker color (e.g. lower transmittance). Layers having a thickness in this range can be formed by pressing, hot pressing, vacuum pressing, tape casting, and the like. In certain examples, the active electrochromic gel can be formed into a solid gel sheet and then laminated between two transparent electrically conductive layers. In other examples, the gel can be prepared with a lower viscosity so that the gel can be poured or spread onto a transparent electrically conductive layer, and then a second transparent electrically conductive layer can be pressed onto the gel layer.

The transparent electrically conductive layers can be made of any material with a higher electrical conductivity compared to the active electrochromic gel layer. In various examples, the transparent electrically conductive layer can have sufficient structural strength to support the electrochromic film. In other examples, the transparent electrically conductive material can be coated onto another transparent substrate that has sufficient structural strength to support the electrochromic film. In a particular example, transparent electrically conductive material can be indium tin oxide. In other examples, the transparent electrically conductive material can include fluorine doped tin oxide (FTO), Poly(3,4-ethylenedioxythiophene) (PEDOT), and the like. In some examples, the transparent electrically conductive layers can have a thickness of 80 nm to 1 μm, and in some cases 100 nanometers to 500 nanometers.

In additional examples, a transparent flexible substrate can be in contact with the first transparent electrically conductive layer. The transparent flexible substrate can include a polymer such as polyethylene terephthalate, or the like. In a specific example, the transparent flexible substrate can be polyethylene terephthalate film and the transparent electrically conductive layer can be indium tin oxide coated on the polyethylene terephthalate film.

The active electrochromic films described herein can also include a sealant to seal in the active electrochromic gel between the transparent electrically conductive layers. The sealant can be applied around a perimeter of the active electrochromic gel. For example, the sealant can be placed between the transparent electrically conductive layers around the edges of the electrochromic film, so that the active electrochromic gel is hermetically sealed in the space between the transparent electrically conductive layers. In another example, the sealant can be applied so that it contains the electrochromic on only a portion of the surface area of the transparent electrically conductive layer. The sealant can be applied in any convenient way, such as applying the sealant to a transparent electrically conductive layer before pressing it to an electrochromic gel layer and a second transparent electrically conductive layer. In another example, the sealant can be applied to edges of the film after the electrochromic gel layer is pressed between two transparent electrically conductive layers. In various examples, the sealant can be a silicone sealant. In other examples, the electrochromic film may not require a sealant for various reasons. For example, the electrochromic gel may be sufficiently viscous that the gel does not escape from between the transparent electrically conductive layers even without a sealant.

In further examples, the active electrochromic films described herein can have an adhesive layer on one side to allow the film to adhere to a substrate such as a window. This can provide an easy method of retrofitting existing windows and other transparent articles. FIG. 2 shows an example active electrochromic film 200 that includes an adhesive layer 250. The film is made up of a transparent flexible substrate 210, a first transparent electrically conductive layer 220, an active electrochromic gel layer 230, a second transparent electrically conductive layer 225, a second transparent flexible substrate 215, and a sealant 240 as in previous examples. However, this example also includes the adhesive layer in contact with the second transparent flexible substrate opposite from the second transparent electrically conductive layer.

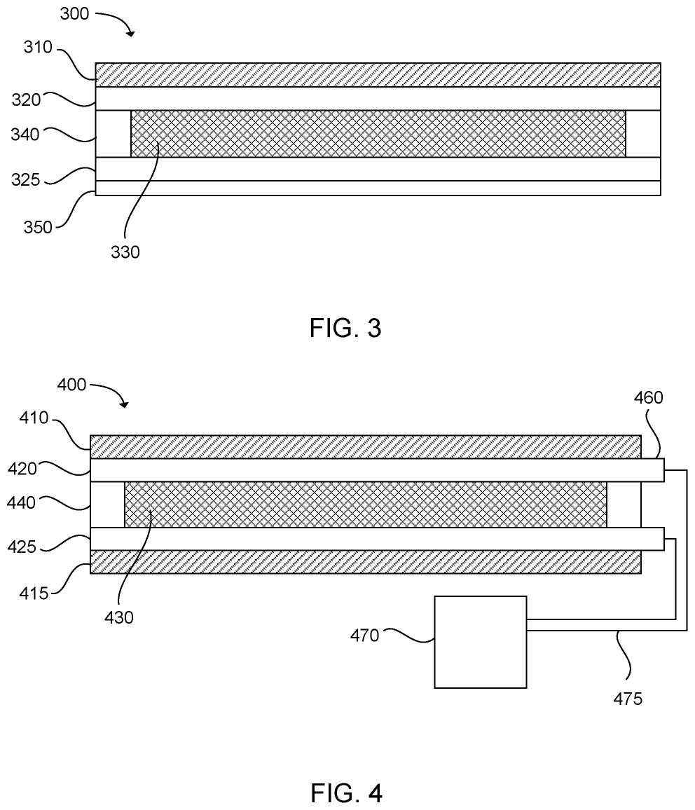

In another example, the active electrochromic film may not include a second transparent flexible substrate, and the adhesive layer can be in direct contact with the second transparent electrically conductive layer. FIG. 3 shows such an example active electrochromic film 300 including a transparent flexible substrate 310, a first transparent electrically conductive layer 320, an active electrochromic gel layer 330, a second transparent electrically conductive layer 325, and a sealant 340. An adhesive layer 350 is in contact with the second transparent electrically conductive layer opposite from the active electrochromic gel layer. The transparent flexible substrate can in some cases provide protection for the transparent electrically conductive layer and active electrochromic gel layer. However, in some examples a protective layer may not be needed on a back surface of the film if the film is to be applied to a window or other rigid structure because the window can protect the transparent electrically conductive layer on the back of the film. Therefore, the adhesive layer can be applied directly to the back transparent electrically conductive layer.

In further examples, the first and second transparent electrically conductive layers can include electrical contacts for connecting the conductive layers to a power supply. The power supply can provide a sufficient voltage to switch the color of the active electrochromic gel layer. FIG. 4 shows another example of an active electrochromic film 400 including a transparent flexible substrate 410, a first transparent electrically conductive layer 420, an active electrochromic gel layer 430, a second transparent electrically conductive layer 425, a second transparent flexible substrate 415, and a sealant 440 as in the examples described above. The first and second transparent electrically conductive layers also include electrical contacts 460 that connect to a power supply 470 through electrical connections 475. In some examples, the power supply can be the electric system of a building or vehicle in which the electrochromic film is installed. In further examples, the power supply can include a battery. Because the electrochromic film can consume a small amount of power, a small battery can be sufficient to operate the electrochromic film. In still further examples, the power supply can include photovoltaic panels. In one example, electrochromic films installed on building windows can be powered by photovoltaic panels that convert sunlight into electric power. This can be particularly beneficial for reducing cooling costs of buildings, as the photovoltaic panels can produce a maximum amount of energy during the part of the day when direct sunlight hits the windows of the building. This time of day naturally corresponds to the most effective time to decrease the transparency of the electrochromic films on the windows to reduce solar heat gain.

The active electrochromic films described herein can also employ masked areas to prevent color switching in certain areas of the films and/or to separate the film into multiple areas that can be color switched independently. In one example, the first transparent electrically conductive layer and/or the second transparent electrically conductive layer can be masked so that the active electrochromic gel layer is insulated from applied voltage in the masked area. An electrically insulating masking material can be placed over the transparent electrically conductive layers before pressing the electrically conductive layers together with the electrochromic gel layer. Thus, the masking material can remain as a part of the film, separating a portion of the electrochromic gel layer from a portion of the transparent electrically conductive layers. In various examples, the masking material can include tape, stickers, masking fluid, nonconductive adhesive, and so on.

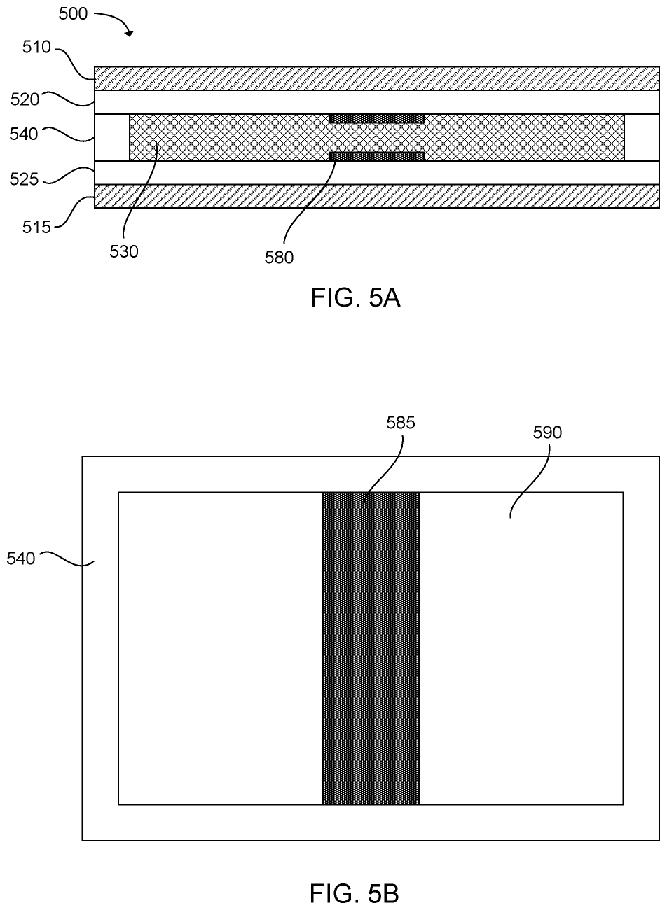

FIG. 5A shows a side cross-sectional view of an example active electrochromic film 500 according to an embodiment of the present technology. The film includes a transparent flexible substrate 510, a first transparent electrically conductive layer 520, an active electrochromic gel layer 530, a second transparent electrically conductive layer 525, a second transparent flexible substrate 515, and a sealant 540 as in the examples described above. Strips of masking material 580 are applied to the first and second transparent electrically conductive layers to insulate the electrochromic gel layer from voltage applied across the transparent electrically conductive layers. FIG. 5B shows a top cross-sectional view of the same film. The masking material covers a masked area 585 and the rest of the area of the transparent electrically conductive layers is an unmasked area 590. In this example, when a voltage is applied across the transparent electrically conductive layers, the electrochromic gel in the unmasked area can switch to a darker color state while the gel in the masked area can remain transparent.

In various examples, the masked and unmasked areas of an active electrochromic film can have any desired shape. The process of applying a masking material to the transparent electrically conductive layers before pressing the electrochromic gel between the layers can make it easy to form simple and complex shapes for the masked area. In some examples, the unmasked portion can be area in which more light blocking is beneficial such as the top of a car windshield, while the masked portion can be an area in which transparency is beneficial, such as the bottom of a car windshield. The masked portion can also be shaped to form images or words, when can be useful for making switchable signage in which the image or words appear when a voltage is applied to the electrochromic film.

In another example, the active electrochromic film can include an additional active electrochromic gel layer and an additional transparent electrically conductive layer aligned with the masked portion. As explained above, the masked portion can remain transparent when a voltage is applied to the first electrochromic gel layer. However, if it is desired to make the masked portion switch to the dark color state as well, then a voltage can be applied to the additional electrochromic gel layer to make this additional layer become dark. Because the additional electrochromic gel layer and the additional transparent electrically conductive layer are aligned with the masked portion, this can cause the film to go dark in the masked area as well.

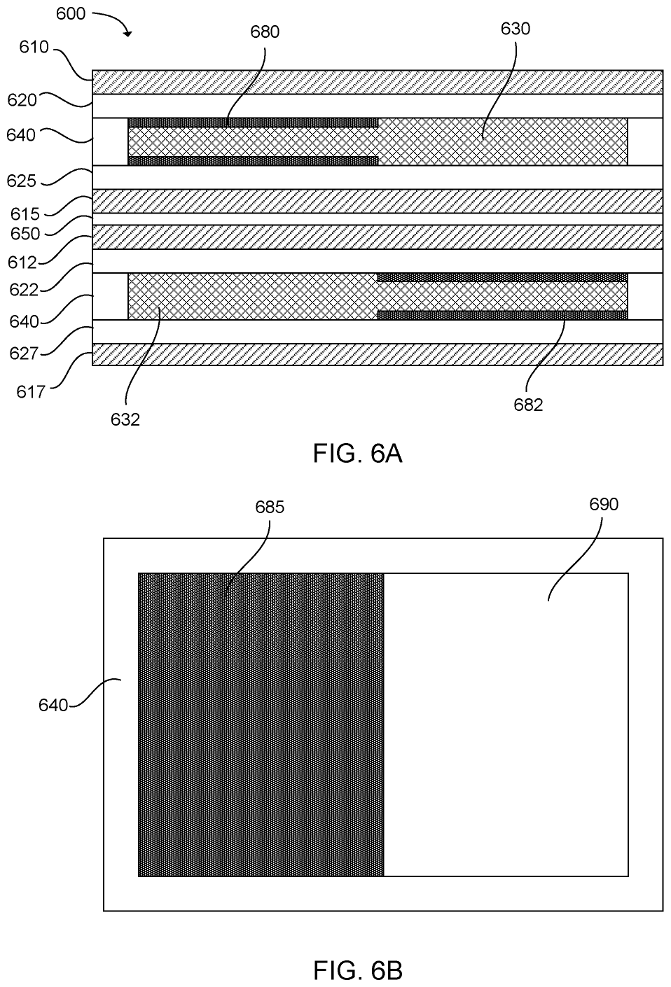

FIG. 6A shows a side cross-sectional view of one such example active electrochromic film 600. This film includes a transparent flexible substrate 610, a first transparent electrically conductive layer 620, an active electrochromic gel layer 630, a second transparent electrically conductive layer 625, a second transparent flexible substrate 615, and a sealant 640 as in the examples described above. Masking material 680 insulates a portion of the active electrochromic gel layer. In this example, an adhesive layer 650 connects the second transparent flexible substrate to a third transparent flexible substrate 612. A similar electrochromic film is structure is formed with the third transparent flexible substrate. The third transparent flexible substrate contacts a third transparent electrically conductive layer 622, which contacts a second active electrochromic gel layer 632. A fourth transparent electrically conductive layer 627 and a fourth transparent flexible substrate 617 are on the opposite surface of the second active electrochromic gel layer. Masking Strips of additional masking material 682 insulate a portion of the second active electrochromic gel layer. FIG. 6B shows a top cross-sectional view of this film, showing the masked area 685 and unmasked area 690 of one of the electrochromic gel layers. The other electrochromic gel layer is masked on the opposite half of the film. In this example, the two electrochromic gel layers are masked in different areas so that two different halves of the film can be independently switched from light to dark by applying voltages to the two electrochromic gel layers. If it is desired to make the entire film go dark, then voltage can be applied to both electrochromic gel layers.

In further examples, any number of electrochromic gel layers can be stacked with various masking patterns to make any number of different areas that can be selectively switched from light to dark. The example shown in FIGS. 6A-6B can be made by forming two essentially compete electrochromic films, each having its own protective transparent flexible substrates, and then joining the two films with an adhesive layer. In further examples, this pattern can be repeated any number of times by adding additional films with additional adhesive layers. In other examples, some of the protective transparent flexible substrates can be omitted because protection may not be necessary in the center of a multi-layer electrochromic film. In one example, an electrochromic film can include a first electrochromic gel layer between transparent electrically conductive layers, and then a second electrochromic gel layer can be applied directly to one of the transparent electrically conductive layers. A third transparent electrically conductive layer can then be applied to the opposite surface of the second electrochromic gel layer. In this example, one or both of the electrochromic gel layers can be activated depending on which pair of transparent electrically conductive layers has an applied voltage. The electrochromic gel layers can be masked in different areas as described above.

Multilayer electrochromic films incorporating masking can be used for a variety of applications. In one example, an electrochromic film for a car windshield can include multiple masked areas so that multiple areas of the windshield can be selectively made darker. This can allow the windshield to be darkened in areas that best reduce glare from sunlight while remaining transparent in areas needed for the driver to see surrounding traffic and the road. As mentioned above, the applied voltage can be varied to adjust the transparency level of the electrochromic gel layers. Thus, the transparency level can be independently adjusted in each unmasked area in the various layers of the multilayer electrochromic film. In certain examples, the multilayer electrochromic film can be connected to a power supply and a controller for controlling which portions of the film are darkened. In one such example, the controller can use light sensors to determine which areas of the film to darken. The controller may incorporate factors such as the angle of the sun in the sky and the position of the driver to determine which areas of the film to darken. Alternatively, the film can have a few portions that can be manually controlled by switches, buttons, or the like so the driver can select which portions of the film to darken.

In further examples, an active electrochromic film can have multiple switchable areas in a single electrochromic gel layer. In one example, the first transparent electrically conductive layer can be divided into at least two electrically isolated portions such that a voltage applied independently from one electrically isolated portion selectively causes a low visible optical transparency in an area of the active electrochromic gel layer adjacent to that electrically isolated portion. In a further example, the first transparent electrically conductive layer can be divided into two electrically isolated portions by masking a portion of the transparent flexible substrate before coating the substrate with the transparent electrically conductive material, and then removing the masking after the coating.

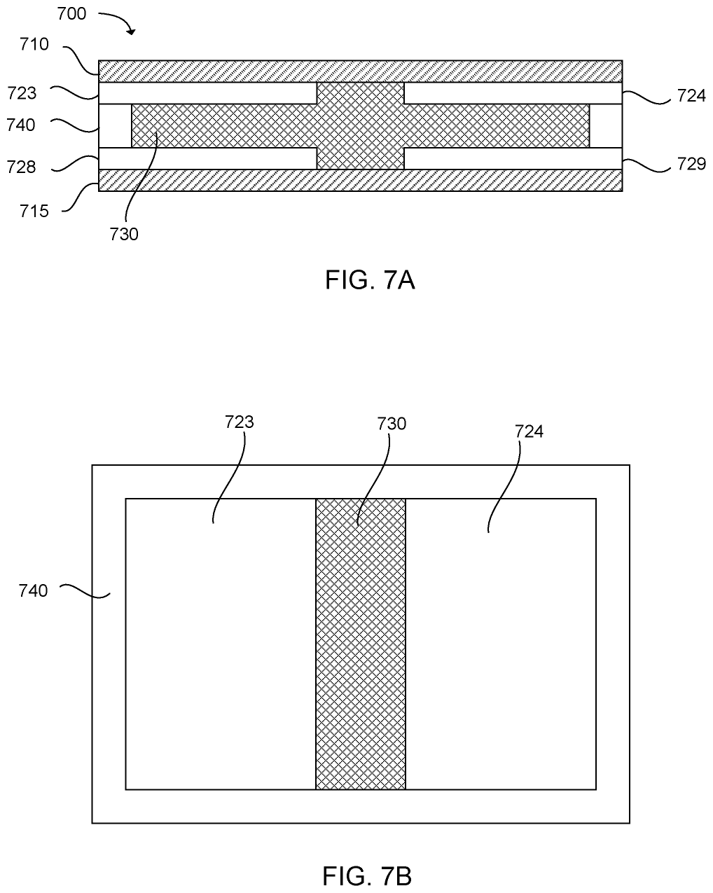

FIG. 7A shows a side cross-sectional view of such an example active electrochromic film 700. This film includes a transparent flexible substrate 710, a first transparent electrically conductive layer divided into electrically isolated portions 723, 724, an active electrochromic gel layer 730, a second transparent electrically conductive layer divided into electrically isolated portions 728, 729, a second transparent flexible substrate 715, and a sealant 740. FIG. 7B shows a top cross-sectional view showing electrically isolated portions 723, 724 with an area of the electrochromic gel layer without any overlapping transparent electrically conductive layers in the center. The electrically isolated portions of the transparent electrically conductive layers can be used to selectively apply voltage to portions of the electrochromic gel layer. In further examples, any number of additional electrically isolated portions of the transparent electrically conductive layers can be formed in any desired shape. Thus, a wide variety of shapes and images can be formed that can be selectively darkened by applying voltage to the electrically isolated portions of the transparent electrically conductive layers.

It should be noted that the figures are not necessarily drawn to scale, and the electrochromic films described herein can have much different dimensions and proportions than shown in the figures. The various components of the electrochromic films have been enlarged and exaggerated in the figures for the purpose of clarity. However, in practice, the various layers in the electrochromic films can be very thin compared to the length and width of the films.

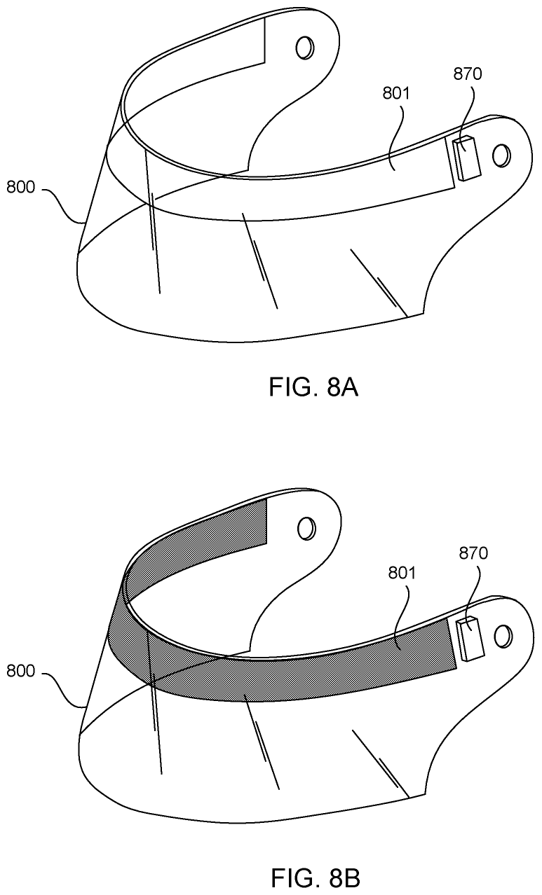

FIG. 8A shows an example of a motorcycle helmet visor 800 having an applied active electrochromic film 801. The visor also includes a power supply 870 to provide a voltage to the film. FIG. 8A shows the film in a transparent state, without an applied voltage. FIG. 8B shows the film in a darkened, powered state. In such examples, the power supply can include a controller to automatically darken the film. In one example, the controller can include a light sensor and the controller can be configured to darken the film when a level of incident light passes a certain threshold. In another example, a simple button or switch can be included to allow a user to darken the film when desired.

The electrochromic films described herein can be made by pressing an active electrochromic gel composition between a first transparent electrically conductive layer and a second transparent electrically conductive layer. In some examples, the first and second transparent electrically conductive layers can be in the form of roll-fed materials. In a particular example, the electrochromic gel compositions can be pressed between roll-fed sheets of polyethylene terephthalate (PET) coated with indium tin oxide (ITO). In other examples, other flexible polymeric substrates and/or other transparent electrically conductive materials can be used.

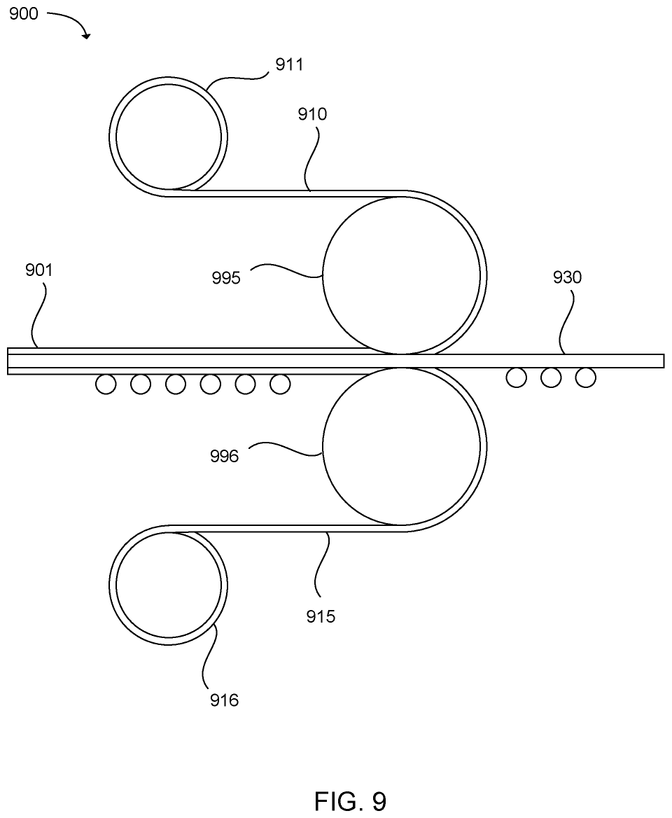

In some examples, the films can be made using hot pressing, vacuum pressing, or the like. FIG. 9 shows a schematic of a system 900 for making an active electrochromic film 901. A sheet of active electrochromic gel composition 930 is fed between two sheets of ITO-coated PET 910, 915. The ITO-coated PET can be fed from rolls 911, 916. These layers can be pressed between heated rollers 995, 996 to form the finished electrochromic film.

### EXAMPLES

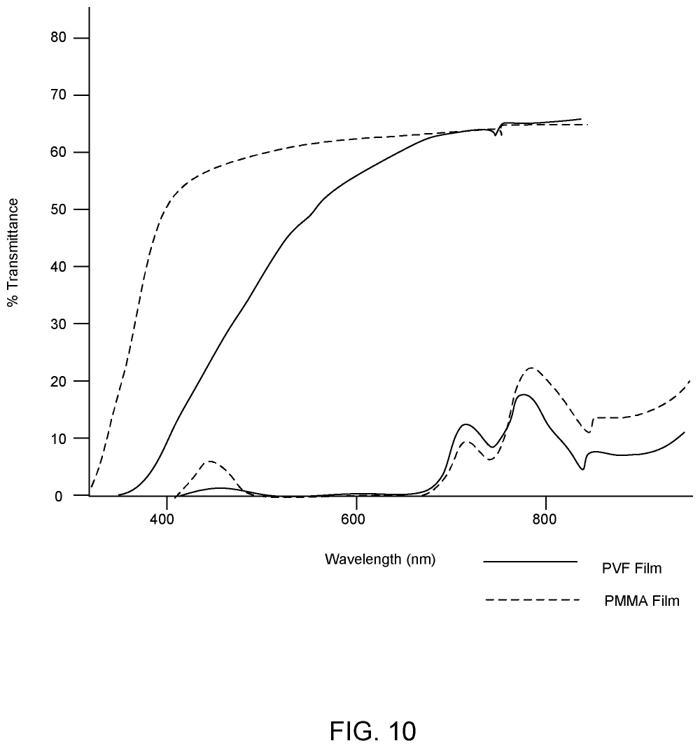

Polymeric viologen gels were prepared by mixing and heating propylene carbonate (PC), hydroquinone, ethyl-viologen, and either polyvinyl-formal (PVF) or poly(methyl methacrylate) (PMMA) diperchlorate. Gel viscosity was controlled by varying PC volume. Electrochromic cells were assembled using the heated viologen gels that were hot-pressed between ITO-coated PET substrates. The films were switched between clear and dark states by applying ±3V and variations in opacity were measured using UV-VIS spectroscopy scanning from 380 to 900 nm. The results of the scan are graphed in FIG. 10. The clear (high transmittance) and dark (low transmittance) states are shown for each film. The PVF film is shown as solid lines, and the PMMA film is shown as dashed lines. The PMMA film generally appeared clearer in the clear state, which is apparent from the higher transmittance of the PMMA film across a broader range of wavelengths. The PVF film had a generally lower transmittance in the dark state, making the film appear darker when powered.

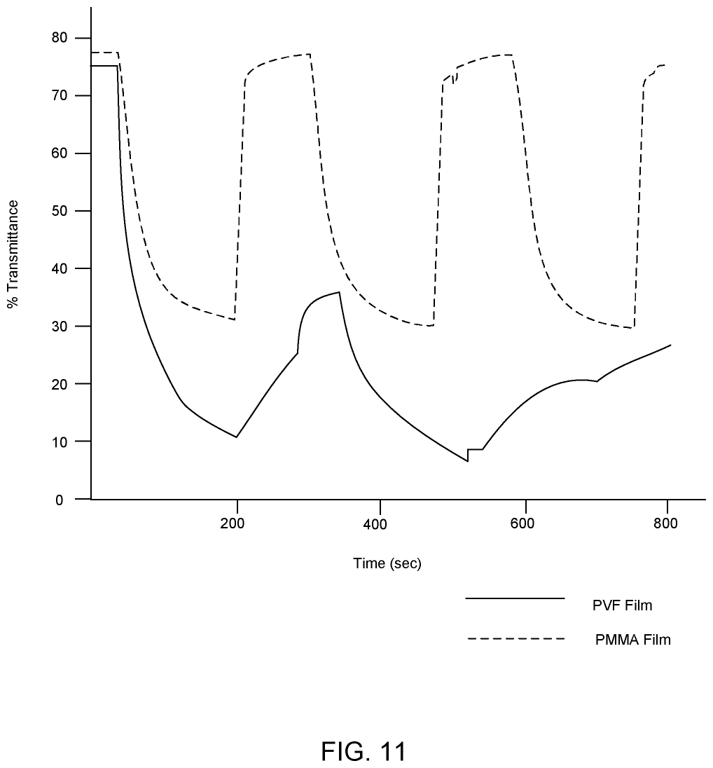

The transition cycle from light to dark was reproducible in both materials, however the PMMA version showed a much more controllable response to switching between light and dark states. FIG. 11 shows the % transmittance of the two films when the films were switched from light to dark several times. The PVF film is shown as a solid line and the PMMA film is shown as a dashed line. The film using PMMA was found to have better transmittance in the clear state with better reproducibility compared to the film using PVF.

It is to be understood that the above-referenced arrangements are illustrative of the application for the principles of the present invention. Thus, while the present invention has been described above in connection with the exemplary embodiments, it will be apparent to those of ordinary skill in the art that numerous modifications and alternative arrangements can be made without departing from the principles and concepts of the invention as set forth in the claims.

1. An active electrochromic film, comprising:
a transparent flexible substrate;
a first transparent electrically conductive layer in contact with the transparent flexible substrate;
an active electrochromic gel layer in contact with the transparent electrically conductive layer, wherein the active electrochromic gel layer comprises a viologen-based compound; and
a second transparent electrically conductive layer in contact with the active electrochromic gel layer opposite from the first transparent electrically conductive layer;
wherein one or both of (a) the active electrochromic gel layer exhibits a high visible optical transparency in the absence of a voltage applied across the first and second transparent electrically conductive layers and a low visible optical transparency under the applied voltage, and wherein at least one of the first and second transparent electrically conductive layers comprises a masked portion such that the active electrochromic gel layer is insulated from the applied voltage in an area adjacent to the masked portion; and (b) the active electrochromic gel layer consists of a homogeneous gel comprising a solvent, a gel-forming polymer, and a viologen-based compound.

2. The active electrochromic film of claim 1, wherein the first transparent electrically conductive layer is divided into at least two electrically isolated portions such that a voltage applied independently from one electrically isolated portion selectively causes a low visible optical transparency in an area of the active electrochromic gel layer adjacent to that electrically isolated portion.

3. The active electrochromic film of claim 1, further comprising an additional active electrochromic gel layer and an additional transparent electrically conductive layer aligned with the masked portion.

4. The active electrochromic film of claim 1, wherein the active electrochromic gel layer consists of a homogeneous gel comprising a solvent and a gel-forming polymer.

5. The active electrochromic film of claim 4, wherein the homogeneous gel has a viscosity of 10,000 cp to 100,000 cp.

6. The active electrochromic film of claim 1, wherein the active electrochromic gel layer further comprises an organic solvent, poly(methyl methacrylate) polymer, and hydroquinone.

7. The active electrochromic film of claim 6, wherein the organic solvent is propylene carbonate.

8. The active electrochromic film of claim 1, wherein the viologen-based compound is ethyl-viologen.

9. The active electrochromic film of claim 1, wherein the active electrochromic gel layer exhibits a high visible optical transparency of at least 70% in the absence of an applied voltage and a low visible optical transparency of less than 50% under the applied voltage.

10. The active electrochromic film of claim 1, wherein the active electrochromic gel layer has a thickness of 0.05 mm to 0.7 mm.

11. The active electrochromic film of claim 1, further comprising a second transparent flexible substrate in contact with the second transparent electrically conductive layer.

12. The active electrochromic film of claim 11, further comprising an adhesive layer in contact with the second transparent flexible substrate.

13. The active electrochromic film of claim 1, further comprising an adhesive layer in contact with the second transparent electrically conductive layer.

14. The active electrochromic film of claim 1, further comprising electrical contacts on the first and second transparent electrically conductive layers connected to a power source to apply a voltage across the active electrochromic gel layer.

15. The active electrochromic film of claim 1, further comprising a sealant between the first and second transparent electrically conductive layers and around a perimeter of the active electrochromic gel layer.

16. The active electrochromic film of claim 1, wherein the transparent flexible substrate comprises polyethylene terephthalate.

17. The active electrochromic film of claim 1, wherein the first transparent electrically conductive layer is indium tin oxide coated on the transparent flexible substrate.

18. (canceled)

19. (canceled)

20. (canceled)

21. (canceled)

22. (canceled)

23. (canceled)

24. (canceled)

25. A method of making an active electrochromic film, comprising:
pressing an active electrochromic gel composition between a first transparent electrically conductive layer and a second transparent electrically conductive layer, wherein the first and second transparent electrically conductive layers are in the form of roll-fed flexible materials and wherein the active electrochromic gel composition comprises a viologen-based compound.

26. The method of claim 25, wherein the pressing is performed by vacuum pressing, heated vacuum pressing, heated rollers, or tape casting.

27. The method of claim 25, wherein the first and second transparent electrically conductive layers are in the form of polyethylene terephthalate coated with indium tin oxide,

28. The method of claim 25, wherein the active electrochromic gel composition is a homogeneous gel comprising a solvent and a gel-forming polymer.

29. The method of claim 25, wherein the active electrochromic gel further comprises an organic solvent, poly(methyl methacrylate) polymer, and hydroquinone.
